(19) United States
(12) Patent Application Publication
(10) Pub. No.: US 2024/0417689 A1
(43) Pub. Date: Dec. 19, 2024

(54) ENGINEERING OF STRATIFIED CELL SHEETS USING MESENCHYMAL STEM CELLS

(71) Applicant: Emmaus Life Sciences, Inc, Torrance, CA (US)

(72) Inventors: Joan Oliva Vilana, Lakewood, CA (US); Jun Ochiai, Kettering, OH (US)

(21) Appl. No.: 18/703,305

(22) PCT Filed: Oct. 21, 2022
(86) PCT No.: PCT/US2022/047381

§ 371 Date: Apr. 19, 2024

Related U.S. Application Data
(60) Provisional application No. 63/360,710, filed on Oct. 21, 2021.

(51) Int. Cl. C12N 5/077; A61L 27/38; C12N 5/00

(52) U.S. Cl. CPC C12N 5/0655; A61L 27/3817; C12N 5/0018; C12N 5/0654; A61L 2300/64; A61L 2430/06; C12N 2501/727; C12N 2506/1384; C12N 2533/90

(57) ABSTRACT

The present disclosure generally relates to the preparation and use of cell sheets. The preparation can entail culturing a plurality of stem cells on a cell culture surface of a container in a culture medium, adding a differentiation medium and a Rho-kinase (ROCK) inhibitor to the container, wherein the differentiation medium and the ROCK inhibitor are added within 2 days of one another. and detaching, from the cell culture surface, a cell sheet formed with cells differentiated from the stem cells. Alternatively, the use of the ROCK inhibitor to remove centrifuge tension can be replaced or supplemented by removal of edges of the cell sheet.

### CROSS REFERENCE TO RELATED APPLICATIONS

This application claims the benefit under 35 U.S.C. § 119(e) of U.S. Provisional Application Ser. No. 63/360,710, filed Oct. 21, 2021, the content of which is incorporated by reference in its entirety into the present disclosure.

### BACKGROUND

There have been reports of work performed with respect to cell sheets. European Patent No. 2266500, for instance, discusses prosthetic tissue or sheet capable of withstanding implantation operations and its production with cells derived from parts other than the myocardium.

European Patent No. 1857126 allegedly provides “a method for producing the cultured cell sheet according to claim 1, which comprises a step of culturing at least one type of cell selected from the group consisting of: chondrocytes, chondroprogenitor cells, synovium derived cells, synovium derived stem cells, osteoblasts, mesenchymal stem cells, adipose derived cells and adipose derived stem cells, on a cell culture support having a surface of the support coated with a temperature responsive polymer having an upper or lower critical solution temperature ranging from 0° C. to 80° C. in water, and thereafter, comprising the steps of: (1) adjusting the temperature of the culture medium to a temperature greater than the upper critical solution temperature or less than the lower critical solution temperature; (2) bringing the cultured cell sheet in close contact with a carrier; and (3) detaching the cultured cell sheet together with the carrier.” Paragraph [0013].

Sato et al., in Regenerative Medicine (2019) 4, allegedly states that “Current cartilage regenerative therapies are not fully effective in treating osteoarthritis of the knee (OAK). We have developed chondrocyte sheets for autologous transplantation and tested these in in vitro and in in vivo preclinical studies, and have reported that the transplantation of chondrocyte sheets promoted hyaline cartilage repair in rat, rabbit, and minipig models. However, autologous transplantation of chondrocyte sheets has yet to be reported in humans. Here we report our combination therapy in which conventional surgical treatment for OAK, is followed by autologous chondrocyte sheet transplantation for cartilage repair.” Abstract.

Thorp et al. in Cells 2021, 10, 643, allegedly state the following: “Articular cartilage defects represent and inciting factor for future osteoarthritis (OA) and degenerative joint disease progression. Despite multiple clinically available therapies that succeed in providing short term pain reduction and restoration of limited mobility current treatments do not reliably regenerate native hyaline cartilage or halt cartilage degeneration at these defect sites. Novel therapeutics aimed at addressing limitations of current clinical cartilage regeneration therapies increasingly focus on allogeneic cells specifically mesenchymal stem cells (MSCs), as potent, banded and available cell sources that express chondrogenic lineage commitment capabilities. Innovative tissue engineering approaches employing allogeneic MSCs aim to develop three-dimensional (3D), chondrogenically differentiated constructs for direct and immediate replacement of hyaline cartilage, improve local site tissue integration, and optimize treatment outcomes. Among emerging tissue engineering technologies, advancements in cell sheet tissue engineering offer promising capabilities for achieving both in vitro hyaline-like differentiation and effective transplantation, based on controlled 3D cellular interactions and retained cellular adhesion molecules.” Abstract.

Despite the various report there is still a need in the art for novel engineering technologies related to the production of cell sheets.

### SUMMARY

In a method aspect, the present disclosure provides a method of producing a cell sheet, using adipose stromal cells, bone marrow stem cells or umbilical cord stem cells. The method, in one embodiments, comprises the steps of: a) contacting a cell culture surface with extracellular matrix to allow cells to attach to the surface; b) seeding cells on the extracellular matrix contacted cell culture surface; c) adding a chemically defined culture medium to the cells; d) culturing the cells at a predetermined temperature under a standard atmosphere; e) monitoring growth of the cell sheet from the cells until a predetermined monitor value has been obtained; f) engineering the cell sheet using mechanical or cell sheet treatment, thereby producing a cell sheet.

In another embodiment, the present disclosure provides a method of preparing a cell sheet, comprising: culturing a plurality of stem cells on a cell culture surface of a container in a culture medium, adding a differentiation medium and a Rho-kinase (ROCK) inhibitor to the container, wherein the differentiation medium and the ROCK inhibitor are added within 2 days of one another, and detaching, from the cell culture surface, a cell sheet formed with cells differentiated from the stem cells.

In another embodiment, provided is a method of producing a cell sheet, comprising: culturing a plurality of stem cells on a cell culture surface of a container in a culture medium, differentiating the stem cells in a differentiation medium, removing cells that grow to become in contact with a wall of the container, and detaching, from the cell culture surface, a cell sheet formed with cells differentiated from the stem cells.

In some embodiments, the stem cells are adipose stromal cells, bone marrow stem cells, mesenchymal stem cells, or umbilical cord stem cells. In some embodiments, the cells differentiated from the stem cells are chondrocytes or osteoblasts.

In some embodiments, the differentiation medium and the ROCK inhibitor are added within 2 days of one another. In some embodiments, the ROCK inhibitor is added within 24 hours after the differentiation medium is added. In some embodiments, the ROCK inhibitor is selected from the group consisting of Y-27632, Y-33075, and H-1152. In some embodiments, the ROCK inhibitor is added to reach a concentration of 1 μM to 20 μM.

Yet another embodiment provides a method of repairing cartilage in a patient who has damaged or unhealthy cartilage, comprising implanting a portion of the cell sheet prepared by a method as disclosed herein at a location within the patient where there is damaged or unhealthy cartilage.

Yet another embodiment provides a method of treating osteoarthritis or an osteochondral defect in a patient who has osteoarthritis, comprising implanting a portion of the cell sheet prepared by a method as disclosed herein at a location within the patient where there is damaged or unhealthy tissue associated with osteoarthritis or where there is an osteochondral defect.

### BRIEF DESCRIPTION OF THE DRAWING

FIG. 1 shows different protocols to engineer cell sheets. The technique can also be used to engineer undifferentiated multilayer cell sheets and osteoblast multilayer cell sheets.

### DETAILED DESCRIPTION

“Cell Sheet” refers to sheet-like clusters of cells that have been grown to confluency and that can be detached from the surface of culture ware as a single sheet (monolayer), or as a multilayer cell sheet (from 2 layers and more).

“Chemically Defined Culture Medium” or “Chemically Defined Medium” refers to a growth medium suitable for the in vitro cell culture of human or animal cells in which all of the chemical components are known. Standard cell culture media (non-chemically defined) commonly consist of a basal medium supplement with animal serum as a source of nutrients and other ill-defined factors.

“Chondrocyte Cell Sheet” refers to a cell sheet comprising chondrocytes. Chondrocytes are the only cells found in healthy cartilage. They produce and maintain the cartilaginous matrix, which consists mainly of collagen and proteoglycans.

“Differentiation” or “Cellular Differentiation” refers to the process where a cell changes from one type to another, oftentimes to a more specialized type.

“Adipose Stromal Cells” refer to multipotent progenitor cells found in adult adipose tissue.

“Bone Marrow Stem Cells” refer to multipotent progenitor cells found in the adult bone marrow.

“Umbilical Cord Stem Cells” refer to multipotent progenitor cells found in the umbilical cord.

“Mesenchymal Stem Cells” refer to multipotent adult stem cells that are present in multiple tissues, including umbilical cord, bone marrow and fat tissue. Mesenchymal stem cells can self-renew by dividing and can differentiate into multiple tissues including bone, cartilage, muscle and fat cells, and connective tissue.

“Osteoblast Cell Sheet” refers to a cell sheet comprising osteoblasts. Osteoblasts are specialized mesenchymal cells that synthesize bone matrix and coordinate the mineralization of the skeleton.

“Rock Inhibitor” is an inhibitor of Rho-associated, coiled-coil containing protein kinase (ROCK). Non-limiting examples of Rock inhibitors include Y-27632 ((1R,4r)-4-((R)-1-aminoethyl)-N-(pyridin-4-yl)cyclohexane-1-carboxamide; CAS No: 129830-38-2), Y-33075 ((R)-4-(1-aminoethyl)-N-(1H-pyrrolo[2,3-b]pyridin-4-yl)benzamide; CAS No: 199433-58-4), H-1152 ((S)-4-methyl-5-((2-methyl-1,4-diazepan-1-yl)sulfonyl)isoquinoline; CAS No: 451462-58-1) and Belumosudil (2-[3-[4-(1H-Indazol-5-ylamino)quinazolin-2-yl]phenoxy]-N-propan-2-ylacetamide; CAS No: 911417-87-3).

In some embodiments, a ROCK inhibitor used herein is selected from Y-27632, Y-33075, H-1152 or Belumosudil. In some embodiments, a ROCK inhibitor used herein is selected from Y-27632, Y-33075, or H-1152. A ROCK inhibitor used herein is selected from Y-27632, or Y-33075.

“Serum-Free Culture Media” refers to a media that does not contain a nutrient and growth factor-rich serum derived from animal blood. Serum-free media uses synthetic or purified ingredients to provide nutrients and growth factors that support growth and survival of cells in culture.

“Stem Cells” refers to cells with the potential to develop into many different types of cells in the body. They serve as a repair system for the body. There are two main types of stem cells: embryonic stem cells and adult stem cells. Stem cells are different from other cells in the body in three ways: they can divide and renew themselves over a long time; they are unspecialized, so they cannot do specific functions in the body; they have the potential to become specialized cells, such as muscle cells, blood cells, and neurons cells as a non-exhaustive list.

“Xeno-Free Culture Media” refers to media that does not contain any ingredient that is either an animal (including human) tissue or body fluid or that is isolated or purified from an animal tissue or body fluid. It may contain recombinant animal or non-animal proteins, including those produced in animal cell lines or by fermentation processes.

“Xenogeneic Compounds” refer to compounds from tissues or cells belonging to individuals of different species.

Chemically defined culture media used in the present disclosure typically include one or more of the following ingredients: water; powder medium; buffering agents; vitamins; lipids; hormones; carrier protein/osmotic; polyamines; attachment factor; antioxidant; transport proteins; growth factors. In certain cases, chemically defined culture media used in the present disclosure include one or more of the following ingredients: water; DMEM/F12 (powder medium); sodium bicarbonate or HEPES (buffering agents); glutamax (amino acid); lipid concentrate (lipids); insulin, hydrocortisone and progesterone (hormones); BSA (carrier protein/osmotic); putrescine (polyamine); Fetuin (attachment factor); Asc-2-P (antioxidant); Holo-transferrin (transport protein); bFGF, EGF, TGF-β1 (growth factors). Nonlimiting examples of commercially available culture media include: KT-016; StemFit for Mesenchymal Stem Cells; MesenCult-ACF Chondrogenic Differentiation Kit; and OsteoMAX-XF Differentiation Media.

In other cases, chemically defined culture media used in the present disclosure comprise the following ingredients: water; DMEM/F12; sodium bicarbonate or HEPES; glutamax; lipid concentrate. In other cases, chemically defined culture media used in the present disclosure comprise the following ingredients: insulin and/or hydrocortisone and/or progesterone; BSA; putrescine; Fetuin; Asc-2-P; Holo-transferrin; bFGF and/or EGF and/or TGF-β1. In other cases, chemically defined culture media used in the present disclosure comprise the following ingredients: water; DMEM/F12; sodium bicarbonate or HEPES; glutamax; lipid concentrate; insulin and/or hydrocortisone and/or progesterone; BSA; putrescine; Fetuin; Asc-2-P; Holo-transferrin; bFGF and/or EGF and/or TGF-β1. In other cases, chemically defined culture media used in the present disclosure comprise: water; DMEM/F12; sodium bicarbonate or HEPES; glutamax; lipid concentrate; insulin, hydrocortisone and progesterone; BSA; putrescine; Fetuin; Asc-2-P; Holo-transferrin; bFGF. EGF and TGF-β1.

### Preparation of Cell Sheets

Cell sheets are useful for tissue regeneration and other therapeutic and commercial uses. A common challenge in the manufacturing of cell sheet is the “centrifuge tension” at the end of the cell culture dish. Centrifuge tension causes cell sheets to detach spontaneously from the cell culture dish, in particular during differentiation of stem cells into chondrocytes. In fact, because of the physical change of the cell sheet into an elastic physical property, the tension of the cell sheet on the edge of the cell culture dish is pulling up and to the edge the cell sheets.

It is discovered herein that centrifuge tension can be eliminated or prevented by cutting the edge of the cell sheet or adding a ROCK inhibitor during cell growth or differentiation.

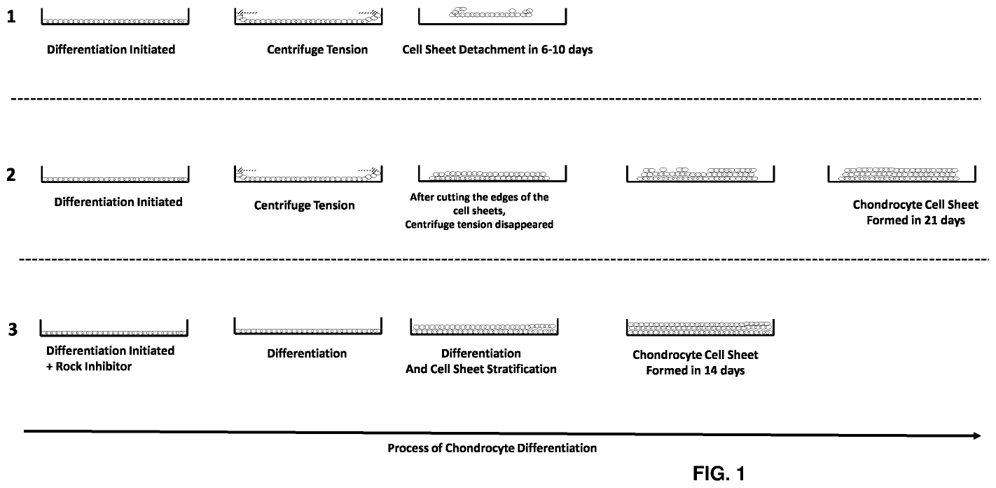

Three different techniques to engineer cell sheets (e.g., chondrocyte cell sheets) are illustrated in FIG. 1. In all three examples (“1”, “2” and “3”), the MesenCult™-ACF Chondrogenic Differentiation Kit can be used to differentiate stem cells (e.g., adipose stromal cells). In “1”, a conventional process, cell sheets detach spontaneously from the cell culture dish due to centrifuge tension. A few days after the beginning of the differentiation, the cell sheets detach, and the cell sheet can't be used for treatment.

In procedure “2”, a technique of the instant disclosure is used. When the edges of the cell sheets start to detach, e.g., at 4-6 days after the initial differentiation state, the edges are cut to detach them from the cell culture dish wall. The cell sheet is left resting in almost absence of culture media to let the cell sheets' edges attach to the cell culture surface (for about 20 minutes). A differentiation culture media (MesenCult™-ACF Chondrogenic Differentiation) is then added to the cell culture dish and a chondrocyte cell sheet is engineered up to 21 days after the initial day of differentiation.

In procedure “3”, a differentiation medium (e.g., MesenCult™-ACF Chondrogenic Differentiation Kit) is supplemented with a Rock Inhibitor (e.g., Y-27632 at 10 μM or higher, Y-33075 or H-1152 at 1 μM or higher). Cell differentiation is be noticed 1 day after initial treatment with the Y-33075 ROCK inhibitor and 2 days after initial treatment with the ROCK inhibitor (Y-27632 and H-1152). Using this methodology, the cell sheets do not detach and are formed in 2 weeks, reducing the time of differentiation by 33%. It was observed that Belumosudil failed to induce stem cell differentiation.

In accordance with one embodiment of the present disclosure, therefore, provided is a method of producing a cell sheet. In one embodiment, the method entails culturing a plurality of cells on a cell culture surface of a container in a culture medium, and removing cells that grow to become in contact with a wall of the container, such that a suitable cell sheet is formed.

In some embodiments, the cells are stem cells which can be differentiated and form a sheet of differentiated cells. Accordingly, in one embodiments, the method entails culturing a plurality of stem cells on a cell culture surface of a container in a culture medium, differentiating the stem cells in a differentiation medium, removing cells that grow to become in contact with a wall of the container, and detaching, from the cell culture surface, a cell sheet formed with cells differentiated from the stem cells.

The removal of the cells on the edge, or alternatively cutting of the edges, can be done with a mechanical tool, such as a knife. In some embodiments, the edge is cut when it touches a wall of the cell culture dish. In some embodiments, the edge is cut when it has started to roll after touching the wall. In some embodiments, the edge is cut before it is in contact with the wall, such as within 1 mm, 2 mm, 3 mm, 4 mm, 5 mm, 6 mm, 7 mm, 8 mm, 9 mm or 10 mm from the wall.

In another embodiment, the ROCK inhibitor is used to inhibit centrifuge tension. An example method entails culturing a plurality of cells on a cell culture surface of a container in a culture medium, and adding a ROCK inhibitor to the medium, such that a suitable cell sheet is formed. Also, such a process can be used for differentiating stem cells and forming a sheet of differentiated cells. Accordingly, in one embodiment, the method entails culturing a plurality of stem cells on a cell culture surface of a container in a culture medium, adding a differentiation medium and a Rho-kinase (ROCK) inhibitor to the container, wherein the differentiation medium and the ROCK inhibitor are added within 2 days of one another, and detaching, from the cell culture surface, a cell sheet formed with cells differentiated from the stem cells.

These two techniques can be combined. That is, if an edge of the cell sheet reaches a wall of the container, the edge can be cut while a ROCK inhibitor is also added to the medium.

In some embodiments, the ROCK inhibitor is added after the differentiation medium is (initially) added. In some embodiments, the ROCK inhibitor is added before the initial differentiation medium. In some embodiments, the addition of both is within 2 days, 24 hours, 18 hours, 12 hours, 8 hours, 6 hours, 4 hours, 2 hours or 1 hour from one another. In a particular embodiment, the ROCK inhibitor is added within 24 hours after the differentiation medium is added.

In some embodiments, the ROCK inhibitor is selected from the group consisting of Y-27632, Y-33075, and H-1152. In some embodiments, the ROCK inhibitor is selected from the group consisting of Y-27632 and Y-33075.

In some embodiments, the final concentration of the ROCK inhibitor in the medium is from 1 μM to 500 μM. In some embodiments, the final concentration is at least 1 μM, 2 μM, 5 μM, 10 μM, 15 μM, 20 μM, 30 μM, 40 μM, 50 μM, 100 μM, 200 μM, or 500 μM. In some embodiments, the final concentration is not higher than 5 μM, 10 μM, 15 μM, 20 μM, 30 μM, 40 μM, 50 μM, 100 μM, 200 μM, or 500 μM.

In some embodiments, the final concentration of Y-27632 in the medium is from 10 μM to 500 82 M. In some embodiments, the final concentration is at least 10 μM, 15 μM, 20 μM, 30 μM, 40 μM, 50 μM, 100 μM, 200 μM, or 500 μM. In some embodiments, the final concentration is not higher than 30 μM, 40 μM, 50 μM, 100 μM, 200 μM, or 500 μM.

In some embodiments, the final concentration of the Y-33075 in the medium is from 1 μM to 500 μM. In some embodiments, the final concentration is at least 1 μM, 2 μM, 5 μM, 10 μM, 15 μM, 20 μM, 30 μM, 40 μM, 50 μM, 100 μM, 200 μM, or 500 μM. In some embodiments, the final concentration is not higher than 5 μM, 10 μM, 15 μM, 20 μM, 30 μM, 40 μM, 50 μM, 100 μM, 200 μM, or 500 μM.

In some embodiments, the final concentration of the H-1152 in the medium is from 1 μM to 500 μM. In some embodiments, the final concentration is at least 1 μM, 2 μM, 5 μM, 10 μM, 15 μM, 20 μM, 30 μM, 40 μM, 50 μM, 100 μM, 200 μM, or 500 μM. In some embodiments, the final concentration is not higher than 5 μM, 10 μM, 15 μM, 20 μM, 30 μM, 40 μM, 50 μM, 100 μM, 200 μM, or 500 μM.

In some embodiments, the number of seeded cells are determined which is useful to determine duration of the differentiation. In some embodiments, the cells seeded in the culture medium, whether stem cells or differentiated cells, start at a relatively high density. For instance, the initial cell density can be ranging from 1,000 cells/cm² to 15,000 cells/cm², or ranging from 2,000 cells/cm² to 15,000 cells/cm², or ranging from 3,000 cells/cm² to 15,000 cells/cm², or ranging from 5,000 cells/cm² to 15,000 cells/cm², or ranging from 7,000 cells/cm² to 15,000 cells/cm². In some embodiments, the initial seeding varies from 26,000 cells/cm² to 104,000 cells/cm². In some embodiments, the initial seeding varies from 10,500 cells/cm² to 550,000 cells/cm².

Different types of cells can be cultured by the process of the present disclosure. In one embodiment, the cells are stem cells, such as adipose stromal cells, bone marrow stem cells, mesenchymal stem cells, and umbilical cord stem cells. During the process, the stem cells may be differentiated, such as into chondrocytes or osteoblasts.

In some embodiments, the differentiation medium includes ingredients (e.g., Osteomax-XF Differentiation Medium (Millipore-Sigma, Burlington, MA)) that promote differentiation of the stem cells to osteoblasts. In some embodiments, the differentiation medium includes ingredients (e.g., MesenCult™-ACF Chondrogenic Differentiation Kit (Stem Cell, Vancouver, Canada)) that promote differentiation of the stem cells to chondrocytes.

In some embodiments, the resulting cell sheet is a monolayer cell sheet. In some embodiments, the resulting cell sheet is a multilayered cell sheet. In some embodiments, the cell sheet includes 2×10⁶ to 20×10⁶ cells per cell sheet, or 4×10⁶ to 10×10⁶ cells per cell sheet, or 5×10⁶ to 7×10⁶ cells per cell sheet. In some embodiments, the cell number, from seeding to cell sheet collection, grows by at least 2-, 3-, 4-, 5-, 6-, 7-, 8-, 9- or 10-fold. In some embodiments, the cell number, from seeding to cell sheet collection, grows by no more than 4-, 5-, 6-, 7-, 8-, 9-, 10-, 15-, or 20-fold.

A cultured undifferentiated cell sheet made (e.g., from 4 to 10 days) using a method of the present disclosure typically expresses phenotype of the original stem cells such as CD73, CD105, Oct¾.

A cultured cell sheet made using a method of the present disclosure typically expresses phenotype of chondroid tissue. In such cases, the cultured undifferentiated cell sheet typically expresses hereditary characteristics such as SOX9, and Oct¾, and toward the differentiation of the cell sheets, the cells express specific markers, which are the characteristics of chondrocyte tissue such as collagen II (ColII), aggrecan (ACAN), secreted protein acidic and rich in cysteine (SPARC), relating to matrix formation which are the characteristics of osteoblast tissue such as osteocalcin (BGLAP), and the absence of HLA-DR for all the cell sheets.

A cultured cell sheet made using a method of the present disclosure is typically constructed of one type of cell, adipose stromal cells or adipose derived cells, but the stratified cell sheets could be engineered with cells from the following, nonlimiting list: chondrocytes, chondroprogenitor cells, synovium derived stem cells, mesenchymal stem cells, adipose derived cells and adipose derived stem cells.

The resulting cell sheet, it has been observed, has excellent biological properties. For instance, cell sheet, following detachment from the preparation cell culture surface, does not shrink more than 25% in either lengthwise direction. In some testing, an undifferentiated cell sheet shrunk by 25.6%, a chondrocyte cell sheet by 6%, and an osteoblast cell sheet shrunk by 8%. In some embodiments, the resulting cell sheet does not shrink more than 40%, 30%, 25%, 20%, 15% 12%, 10%, 8%, or 5% in either lengthwise direction.

Cell sheets made using a method according to the present disclosure are multilayer cell sheets. Multilayer cell sheets are typically comprised of at least two layers, with undetermined upper limits. In the present disclosure, cell sheets with 11 layers have been produced, with an average of 6 layers.

In an example aspect, a method of producing monolayer cell sheets or multilayer cell sheets is provided. A cell culture surface (e.g., culture dish) is obtained, but the surface is already pre-treated with extracellular matrix proteins. The extracellular matrix is brought in contact with the cell culture surface to allow cells to attach to the surface. The cell culture surface is not treated with serum, polymer or otherwise modified. Cells are seeded on the cell culture surface to a particular density as disclosed. A chemically defined culture medium is added to the cells, which is replaced at predetermined intervals, until the cells reach confluence. A chemically defined culture medium, for cells differentiation, is used from that moment. The cells are cultured at a predetermined temperature (e.g., 37° C.) under a standard atmosphere (e.g., 5% CO₂). Cell sheet growth is monitored, typically using transmittance values measured over time and/or by visual inspection using an inverted microscope.

For undifferentiated multilayer cell sheets and for osteoblast multilayers cell sheets, culture media changes can be the only requirement. In certain cases, engineering the multilayer cell sheets involves cutting the edge of the cell sheets, or treating the cell sheets with ROCK inhibitor (e.g., Rock Inhibitor Y-27632, Y-33075), especially when the cell sheet is becoming more “elastic” and involves centrifuges forces pulling the cell sheet and leading to its detachment (FIG. 1). Cutting the edges of the cell sheets or using ROCK inhibitor alleviates the centrifuges forces that pulls and detach the cell sheet during the culturing time.

ROCK 1 and 2 inhibitors (e.g., Y-27632 and Y-33075) were used with success in engineering the chondrocyte multilayer cell sheets. Differentiation of the cells started at 1 and 2 days, and it is dose dependent, for Y-33075 and Y-27632 respectively. The use of ROCK 2 inhibitor H-1152 induced the cell differentiation but it is less efficient than Y-27632 and Y-33075. On the contrary, Belumosudil does not induce cell differentiation and it is toxic for the cells in a dose response, inducing cell death.

Harvesting of the cell sheets can be performed once a predetermined monitor value (e.g., transmittance) has been obtained. Harvesting is typically performed using mechanical means. In other words, mechanical harvesting typically does not involve temperature treatment (e.g., low temperature treatment), enzymatic methods, electro-responsive, photo-responsive, pH responsive, magnetic, surface. Mechanical harvesting involves lifting the cell sheet with a mechanical tool (e.g., forceps) after placing or not a membrane over the cell sheet. Oftentimes, the harvested cell sheet is transferred to a carrier (e.g., formed from polyvinylidene difluoride film) prior to further use, such as use in tissue (e.g., cartilage) reconstruction, or the cell sheet can be transplanted directly on the tissue (e.g., cornea).

### Therapeutic Uses of the Cell Sheets

Cell sheets made using a method of the present disclosure can be used in tissue reconstruction. Cell sheets may be used, for example, for the repair/regeneration of cartilage, bone or the bone-cartilage complex. Multilayered chondrocyte cell sheets are oftentimes of particular use, due to high expression levels of chondrogenic-related genes and proteins, as well as of cell adhesion-related genes and proteins. Such multilayer cell sheets are also able to secret certain factors that play a role in cartilage regeneration. Tissue reconstruction using multilayered chondrocyte cell sheets can also be used to treat osteoarthritis, where there is the destruction of cartilage and subchondral bone.

In one embodiment, a method is provided for repairing cartilage in a patient who has damaged or unhealthy cartilage. The method includes implanting a portion of the cell sheet prepared by a method of the present disclosure at a location within the patient where there is damaged or unhealthy cartilage.

Also provided, in one embodiment, is a method of treating osteoarthritis or an osteochondral defect in a patient who has osteoarthritis, which entails implanting a portion of the cell sheet prepared by a method of the present disclosure at a location within the patient where there is damaged or unhealthy tissue associated with osteoarthritis or where there is an osteochondral defect.

In some embodiments, the method further entails treating the patient with a surgical treatment to remove damaged or unhealthy cartilage or osteochondral defect before the implanting.

The cells may be autologous cells or allogeneic cells. In some embodiments, the cell sheet includes chondrocytes.

Methods of producing cell sheets according to the present disclosure reduce the cost of manufacturing given that the methods are more reproducible and result in usable cell sheets at a much greater rate. For instance, using the present methods, one typically obtains cell sheets that can be used in a treatment modality between 60 percent and 100 percent of the time. Oftentimes, usable cell sheets are obtained between 70 percent and 100 percent of the time, between 80 percent and 100 percent of the time, or between 90 percent and 100 percent of the time.

Given that the cost for GMP facility use is typically around $50,000/week, and that manufacturing can take on the order of months (e.g., 1 month, two months, three months or six months), this increased reproducibility can save between $50,000 and $500,000 in the development of cell sheet product. In certain cases, it can save between $50,000 and $1,000,000, between $50,000 and $1,500,000, or between $50,000 and $2,000,000. The amount of savings is calculated using methods discussed in Ham et al. “What does cell therapy manufacturing cost? A framework and methodology to facilitate academic and other small-scale therapy manufacturing costings” Cytotherapy, Vol. 22, Issue 7, Jul. 2020, pages 388-397, which is incorporated into this document for all purposes.

### EXPERIMENTAL EXAMPLES

### Example 1. Preparation of Cell Sheets

Human adipose stromal cells (ASC) were purchased from RoosterBio, Inc. (RoosterBio, Inc., Frederick, MD). Human ASC was used for the following experiments. hASC were expanded up to passage 4 or 5, in T75 flask (USAScientific, Ocala, FL), using RoosterNourish™-MSC-XF (RoosterBio, Inc., Frederick, MD) or using Stem Fit for Mesenchymal Stem Cells (Ajinomoto, Tokyo, Japan).

Umbilical Cord Stem Cells (UCSC) and Bone Marrow Stem Cells (BMSC) were purchased from RoosterBio, Inc (RoosterBio, Inc., Frederick, MD). UCSC and BMSC were expanded up to passage 3, in flask pre-treated with vitronectin (0.25 μg/cm²).

ASC were seeded at 1.05, 2.6, 5.2, 11.3, 22.1 and 55×10⁴ ASC per cm², in 35 mm culture dish (Corning, Corning, NY). The ASC were cultured with RoosterNourish™-MSC-XF culture media (RoosterBio, Inc., Frederick, MD) or with Stem Fit for Mesenchymal Stem Cells (Ajinomoto, Tokyo, Japan). All engineered cell sheets were multilayer when they were harvested. Undifferentiated ASCCS: ASC were cultured with RoosterNourish™M-MSC-XF. The culture media was replaced every 2 days, up to 12 days from the initial seeding day. Multilayer undifferentiated cell sheets were engineered and harvested, regardless the initial cell seeding. Cell sheet engineered with the initial seeding at 1.05×10⁴ ASC per cm² in 12 days.

ASC were seeded at 11.3×10⁴ ASC per cm², in 35 mm culture dish (Corning, Corning, NY). The ASC were cultured with RoosterNourish™-MSC-XF culture media (RoosterBio, Inc., Frederick, MD) or with Stem Fit for Mesenchymal Stem Cells (Ajinomoto, Tokyo, Japan). All engineered cell sheets were multilayer when they were harvested.

Undifferentiated ASCCS: ASC were cultured with RoosterNourishT™-MSC-XF. The culture media was replaced every 2 days, up to 10 days from the initial seeding day. The same protocol was used when the ASC were cultured with StemFit.

Osteoblast Cell Sheet: ASC were cultured with RoosterNourish™-MSC-XF, until the cells reached confluence. From that day, ASC were cultured with Osteomax-XF Differentiation Medium (Millipore-Sigma, Burlington, MA). The culture media was replaced every 3 days, up to 17 days from the initial seeding day.

Chondrocyte Cell Sheet: ASC were cultured with RoosterNourish™-MSC-XF, until the cells reached confluence. From that day, ASC were cultured with MesenCult™-ACF Chondrogenic Differentiation Kit (Stem Cell, Vancouver, Canada). The culture media was replaced every 2 days, up to X days from the initial seeding day depending on the technique used to engineer the cell sheet.

One of two techniques was used to complete the engineering process: 1) To stop centrifuge tension, the edges of the cell sheets were cut, and the cell sheet was left resting in almost absence of culture media (MesenCult™-ACF Chondrogenic Differentiation) to let the cell sheets' edges attached to the cell culture surface, for 20 minutes. The differentiation culture media (MesenCult™-ACF Chondrogenic Differentiation) was added to the cell culture dish and the chondrocyte cell sheet was engineered up to 21 days after the initial day of differentiation. 2) ROCK inhibitor Y-27632 at 10 μM or ROCK inhibitor Y-33075 at 1 μM were used from the first day of differentiation. Using this methodology, the cell sheets didn't detach and were formed in 2 weeks, reducing by 33% the time of differentiation.

### Example 2. Examination of Cell Sheets

Human adipose stromal cells were used to engineer cell sheets. The cells were seeded at 11.3×10⁴ ASC per cm². Chemically defined culture media were used to culture and engineer the cell sheets. A device, measuring the transmittance of the engineered cell sheet, was used to estimate the time for harvesting. Total transcriptome of the cell sheets was analyzed with microarray, and the data were confirmed with real-time PCR and immunohistochemistry.

Undifferentiated ASCCS were engineered in 10 days, using a chemically defined culture media. Differentiated ASCCS into Chondrocyte (treated with ROCK inhibitor from 1 to 20 μM), chondrocyte and osteoblast were also engineered using chemically defined and animal free culture media, in 14, 21 and 14 days, respectively. The transmittance of the ASCCS were measured over time from the time the cells reached confluence until the cell sheets were harvested. The transmittance decreased over time for all the cell sheets, at different rates. In addition, the transmittance value was lower for the osteoblast cell sheets compared to the other cell sheets confirming their visual observation.

The mechanical harvesting of the cell sheets was an easy process because of the strong cell-cell connection and the stratification of the cell sheets. Real time qPCR and IHC confirmed the identity of the cell sheets: CD19-, CD45R-, HLA-DR-, HLA-, CD29+, CD73+, CD105+for the undifferentiated cell sheets; bone gamma-carboxyglutamic acid-containing protein was present in osteoblast cell sheet; aggrecan core protein, secreted protein acidic and cysteine rich and collagen type II in the chondrocyte cell sheet. The differentiation was also confirmed with cell sheet dye using alcian blue (for chondrocyte cell sheet) and alizarin red (osteoblast cell sheet). RNA, analyzed by microarray, can provide detailed information about the cell sheet transcriptome, confirming the presence of proteins involved in the cell-cell connection (e.g., connexins).

Different stratified cell sheets were engineered successfully using human adipose stromal stem cells, with chemically defined culture media. Adipose stromal cells preserved their differentiation potential and differentiated cell sheets expressed specific markers of targeted tissues. The absence of xeno-products and animal/human serum could decrease the potential risk of xeno-contamination of patients. In addition, it increases the reproducibility of cell sheet engineering by eliminating variability among animal serum lots.

### REFERENCES

1. Gegg, C.; Yang, F., The Effects of ROCK Inhibition on Mesenchymal Stem Cell Chondrogenesis Are Culture Model Dependent. Tissue Eng Part A 2020, 26, (3-4), 130-139.
2. Wang, K. C.; Egelhoff, T. T.; Caplan, A. I.; Welter, J. F.; Baskaran, H., ROCK Inhibition Promotes the Development of Chondrogenic Tissue by Improved Mass Transport. Tissue Eng Part A 2018, 24, (15-16), 1218-1227.
3. Thorp, H.; Kim, K.; Kondo, M.; Grainger, D. W.; Okano, T., Fabrication of hyaline-like cartilage constructs using mesenchymal stem cell sheets. Sci Rep 2020, 10, (1), 20869.
4. Sato, M.; Yamato, M.; Mitani, G.; Takagaki, T.; Hamahashi, K.; Nakamura, Y.; Ishihara, M.; Matoba, R.; Kobayashi, H.; Okano, T.; Mochida, J.; Watanabe, M., Combined surgery and chondrocyte cell-sheet transplantation improves clinical and structural outcomes in knee osteoarthritis. NPJ Regen Med 2019, 4, 4.
5. Kaneshiro, N.; Sato, M.; Ishihara, M.; Mitani, G.; Sakai, H.; Kikuchi, T.; Mochida, J., Cultured articular chondrocytes sheets for partial thickness cartilage defects utilizing temperature-responsive culture dishes. Eur Cell Mater 2007, 13, 87-92.
6. Lu, Y.; Zhang, W.; Wang, J.; Yang, G.; Yin, S.; Tang, T.; Yu, C.; Jiang, X., Recent advances in cell sheet technology for bone and cartilage regeneration: from preparation to application. Int J Oral Sci 2019, 11, (2), 17.
7. Akahane, M.; Nakamura, A.; Ohgushi, H.; Shigematsu, H.; Dohi, Y.; Takakura, Y., Osteogenic matrix sheet-cell transplantation using osteoblastic cell sheet resulted in bone formation without scaffold at an ectopic site. J Tissue Eng Regen Med 2008, 2, (4), 196-201.
8. Ueyama, Y.; Yagyuu, T.; Maeda, M.; Imada, M.; Akahane, M.; Kawate, K.; Tanaka, Y.; Kirita, T., Maxillofacial bone regeneration with osteogenic matrix cell sheets: An experimental study in rats. Arch Oral Biol 2016, 72, 138-145.
9. Nakamura, A.; Akahane, M.; Shigematsu, H.; Tadokoro, M.; Morita, Y.; Ohgushi, H.; Dohi, Y.; Imamura, T.; Tanaka, Y., Cell sheet transplantation of cultured mesenchymal stem cells enhances bone formation in a rat nonunion model. Bone 2010, 46, (2), 418-24.
10. Ma, D.; Ren, L.; Liu, Y.; Chen, F.; Zhang, J.; Xue, Z.; Mao, T., Engineering scaffold-free bone tissue using bone marrow stromal cell sheets. J Orthop Res 2010, 28, (5), 697-702.
11. Bjare, U., Serum-free cell culture. Pharmacol Ther 1992, 53, (3), 355-74.
12. Kimberlin, R. H., Introduction to scrapie and perspectives on current scrapie research. Prog Clin Biol Res 1989, 317, 559-66.
13. Wappler, J.; Rath, B.; Laufer, T.; Heidenreich, A.; Montzka, K., Eliminating the need of serum testing using low serum culture conditions for human bone marrow-derived mesenchymal stromal cell expansion. Biomed Eng Online 2013, 12, 15.
14. Cimino, M.; Goncalves, R. M.; Barrias, C. C.; Martins, M. C. L., Xeno-Free Strategies for Safe Human Mesenchymal Stem/Stromal Cell Expansion: Supplements and Coatings. Stem Cells Int 2017, 2017, 6597815.
15. Mabry, R.; Brasky, K.; Geiger, R.; Carrion, R., Jr.; Hubbard, G. B.; Leppla, S.; Patterson, J. L.; Georgiou, G.; Iverson, B. L., Detection of anthrax toxin in the serum of animals infected with Bacillus anthracis by using engineered immunoassays. Clin Vaccine Immunol 2006, 13, (6), 671-7.
16. Ryan, E. D.; Kirby, M.; Collins, D. M.; Sayers, R.; Mee, J. F.; Clegg, T., Prevalence of Coxiella burnetii (Q fever) antibodies in bovine serum and bulk-milk samples. Epidemiol Infect 2011, 139,(9), 1413-7.
17. Zabal, O.; Kobrak, A. L.; Lager, I. A.; Schudel, A. A.; Weber, E. L., [Contamination of bovine fetal serum with bovine viral diarrhea virus]. Rev Argent Microbiol 2000, 32, (1), 27-32.
18. Knepper, P. A.; Mayanil, C. S.; Goossens, W.; McLone, D. C.; Hayes, E., The presence of transcription factors in fetal bovine sera. In Vitro Cell Dev Biol Anim 1998, 34, (2), 170-3.
19. Dominici, M.; Le Blanc, K.; Mueller, I.; Slaper-Cortenbach, I.; Marini, F.; Krause, D.; Deans, R.; Keating, A.; Prockop, D.; Horwitz, E., Minimal criteria for defining multipotent mesenchymal stromal cells. The International Society for Cellular Therapy position statement. Cytotherapy 2006, 8, (4), 315-7.
20. Murphy, L. B.; Cisternas, M. G.; Pasta, D. J.; Helmick, C. G.; Yelin, E. H., Medical Expenditures and Earnings Losses Among US Adults With Arthritis in 2013. Arthritis Care Res (Hoboken) 2018, 70, (6), 869-876.

1. A method of preparing a cell sheet, comprising:
culturing a plurality of stem cells on a cell culture surface of a container in a culture medium,
adding a differentiation medium and a Rho-kinase (ROCK) inhibitor to the container, wherein the differentiation medium and the ROCK inhibitor are added within 2 days of one another, and
detaching, from the cell culture surface, a cell sheet formed with cells differentiated from the stem cells.

2. The method of claim 1, wherein the cell culture surface is coated with extracellular matrix.

3. The method of claim 1, wherein the culture medium is a chemically defined culture medium.

4. The method of claim 1, further comprising counting the number of stem cells prior to addition of the differentiation medium, wherein the counted number is used to determine duration of the differentiation.

5. The method of claim 1, wherein the detaching is with a mechanical tool.

6. The method of claim 1, further comprising removing cells that grow to be in contact with a wall of the container.

7. The method of claim 6, wherein the removing comprises cutting edges of the cell sheet with a mechanical tool.

8. The method of claim 1, wherein the stem cells are selected from the group consisting of adipose stromal cells, bone marrow stem cells, mesenchymal stem cells, and umbilical cord stem cells.

9. The method of claim 1, wherein the cells differentiated from the stem cells are chondrocytes or osteoblasts.

10. The method of claim 1, wherein the differentiation medium and the ROCK inhibitor are added within 1 day of one another.

11. The method of claim 1, wherein the ROCK inhibitor is added within 24 hours after the differentiation medium is added.

12. The method of claim 1, wherein the ROCK inhibitor is selected from the group consisting of Y-27632, Y-33075, and H-1152.

13. The method of claim 1, wherein the ROCK inhibitor is added to reach a concentration of 1 μM to 20 μM.

14. The method of claim 1, wherein the cell sheet includes 5×10⁶ to 7×10⁶ cells.

15. The method of claim 1, wherein the cell sheet is a multilayered cell sheet.

16. The method of claim 1, wherein the cell sheet, following detachment, does not shrink more than 20% in either lengthwise direction.

17. A method of producing a cell sheet, comprising:
culturing a plurality of stem cells on a cell culture surface of a container in a culture medium,
differentiating the stem cells in a differentiation medium,
removing cells that grow to become in contact with a wall of the container, and
detaching, from the cell culture surface, a cell sheet formed with cells differentiated from the stem cells.

18. The method of claim 17, wherein the removing comprises cutting edges of the cell sheet with a mechanical tool.

19. The method of claim 17, further comprising adding a Rho-kinase (ROCK) inhibitor to the container, wherein the differentiation medium and the ROCK inhibitor are added within 2 days of one another.

20-24. (canceled)

25. A method of repairing cartilage in a patient who has damaged or unhealthy cartilage, comprising implanting a portion of the cell sheet prepared by a method of claim 1 at a location within the patient where there is damaged or unhealthy cartilage.

26-29. (canceled)
